(19) United States
(12) Patent Application Publication
(10) Pub. No.: US 2026/0027087 A1
(43) Pub. Date: Jan. 29, 2026

(54) DOSING REGIMENS COMPRISING A KAT6 INHIBITOR FOR THE TREATMENT OF CANCER

(71) Applicants: PFIZER INC., New York, NY (US); CTXT PTY LTD, Parkville (AU)

(72) Inventors: Eric Chris GREENWALD, San Diego, CA (US); Natasha Farrokh HOMJI MISHRA, San Diego, CA (US); Karen Leann KOWLASKI, San Diego, CA (US); Meng LI, San Diego, CA (US); Li LIU, San Diego, CA (US); Jingwen ZHENG, Thousand Oaks, CA (US)

(21) Appl. No.: 19/099,067

(22) PCT Filed: Jul. 25, 2023
(86) PCT No.: PCT/IB2023/057537

§ 371 Date: Jan. 28, 2025

Related U.S. Application Data
(60) Provisional application No. 63/393,504, filed on Jul. 29, 2022.
(60) Provisional application No. 63/468,677, filed on May 24, 2023.
(60) Provisional application No. 63/470,365, filed on Jun. 1, 2023.

(51) Int. Cl. A61K 31/423; A61K 31/4196; A61K 31/506; A61K 31/519; A61K 31/565; A61P 35/00

(52) U.S. Cl. CPC A61K 31/423; A61K 31/4196; A61K 31/506; A61K 31/519; A61K 31/565; A61P 35/00

(57) ABSTRACT

The invention relates to dosing regimens for the treatment of cancer comprising administering to a subject in need thereof a daily dose of a lysine acetyltransferase 6 (KAT6) inhibitor as a single agent or in combination with: a) a cyclin-dependent kinase 4 (CDK4) inhibitor; b) an antiestrogen; or c) a CDK4 inhibitor and an antiestrogen.

### BACKGROUND OF THE INVENTION

KAT6A and KAT6B are histone lysine acetyltransferases that acetylate H3K23, and their enzymatic functions are involved in fundamental cellular processes, including gene transcription, cellular senescence, tissue development, and maintenance of normal hematopoietic stem cells (Huang, F., et al., Regulation of KAT6 Acetyltransferases and Their Roles in Cell Cycle Progression, Stem Cell Maintenance, and Human Disease. Mol Cell Biol. 2016, 36(14):1900-7). KAT6A has been implicated in promoting tumorigenesis in a variety of cancers with KAT6A amplifications and over-expression observed in breast cancer, prostate cancer, ovarian cancer, uterine cervix cancer, lung adenocarcinoma, colon & rectal adenocarcinomas and medulloblastoma (Yu, L., et al., Identification of MYST3 as a novel epigenetic activator of ERα frequently amplified in breast cancer. Oncogene 2017, 36(20):2910-8; Tsherniak, A., et al., Defining a cancer dependency map. Cell 2017; 170(3)(July):564-576.e16; Zack, T. I., et al., Pan-cancer patterns of somatic copy number alteration. Nat Genet. 2013, 45:1134-1140; and Northcott, P. A., et al., Multiple recurrent genetic events converge on control of histone lysine methylation in medulloblastoma. Nat Genet. 2009, 41(4):465-72). KAT6A chromosomal translocations have been observed in AML (See Huang F, et al.; Borrow, J., et al., The translocation t(8;16)(p11;p13) of acute myeloid leukaemia fuses a putative acetyltransferase to the CREB-binding protein. Nat. Genet. 1996; 14(1):33-41; and Shima, H., et al., Bromodomain-PHD finger protein 1 is critical for leukemogenesis associated with MOZ-TIF2 fusion. Int J Hematol. 2014; 99(1):21-31). KAT6 inhibition has therapeutic potential in multiple disease settings, including breast, prostate and NSCLC.

2-Methoxy-N-{4-methoxy-6-[(1H-pyrazol-1-yl)methyl]-1,2-benzoxazol-3-yl}benzene-1-sulfonamide (referred to herein as “COMPOUND A”, “Compound A” or “COMPD A”), is a potent and selective catalytic inhibitor of KAT6 histone acetyltransferases, KAT6A and KAT6B. COMPOUND A is currently in phase I clinical trials for the treatment of cancers, and has the following structure:

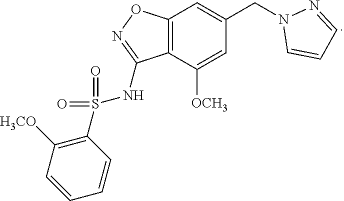 [SMILES: COc1ccccc1S(=O)(=O)Nc1noc2cc(Cn3cccn3)cc(OC)c12]

Preparation of COMPOUND A, including an anhydrous crystalline form of COMPOUND A free acid, is described in International Publication No. WO 2020/254946. Combination therapies including COMPOUND A are described in International Patent Publication No. WO 2022/013369. The contents of each of the foregoing documents are incorporated herein by reference in their entirety.

Cyclin-dependent kinases (CDKs) and related serine/threonine protein kinases are important cellular enzymes that perform essential functions in regulating eukaryotic cell division and proliferation. The CDK catalytic units are activated by regulatory subunits known as cyclins. At least sixteen mammalian cyclins have been identified (Johnson D G, Walker C L. Cyclins and Cell Cycle Checkpoints. Annu. Rev. Pharmacol. Toxicol. (1999) 39:295-312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation and apoptosis (Morgan D O, Cyclin-dependent kinases: engines, clocks, and microprocessors. Annu. Rev. Cell. Dev. Biol. (1997) 13:261-291).

CDK inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp J E, Broder S. Molecular foundations of cancer: new targets for intervention. Nat. Med. (1995) 1:309-320; and Hall M, Peters G. Genetic alterations of cyclins, cyclin-dependent kinases, and Cdk inhibitors in human cancer. Adv. Cancer Res. (1996) 68:67-108).

CDK4 and CDK6 are important regulators of cell cycle progression at the G1-S checkpoint, which are controlled by D-type cyclins and INK4 endogenous CDK inhibitors, such as p16^{INK4a} (CDKN2A). Dysregulation of the cyclin D-CDK4/6-INK4-retinoblastoma (Rb) pathway has been reported to be associated with development of endocrine therapy resistance. Furthermore, CDK4 has been identified as the singular oncogenic driver in many breast cancers and emerging data suggest that cyclin D3-CDK6 inhibition may be linked to hematologic toxicity, suggesting a role for CDK4 selective inhibitors.

Clinical trials for the CDK4/6 inhibitors palbociclib, ribociclib and abemaciclib are ongoing for breast and other cancers, as single agents or in combination with other therapeutics. The use of CDK4/6 inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors, including palbociclib, ribociclib and abemaciclib, have been approved in combination with endocrine therapy in a first- or second-line setting. Palbociclib, ribociclib and abemaciclib have been approved for treatment of hormone receptor (HR)-positive, human epidermal growth factor receptor 2 (HER2)-negative advanced or metastatic breast cancer in combination with aromatase inhibitors, such as letrozole, in a first line setting and with fulvestrant in second or later lines of therapy in certain patients. (O'Leary et al. Treating cancer with selective CDK4/6 inhibitors. Nature Reviews (2016) 13:417-430).

Palbociclib, or 6-acetyl-8-cyclopentyl-5-methyl-2-(5-piperazin-1-yl-pyridin-2-ylamino)-8H-pyrido[2,3-d]pyrimidin-7-one (also referred to as “PD-0332991”) is a potent and selective inhibitor of CDK4 and CDK6, having the structure:

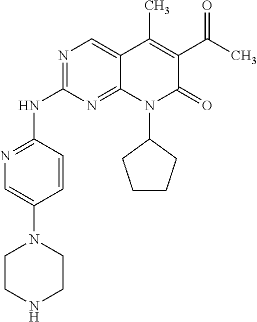 [SMILES: CC(=O)c1c(C)c2cnc(Nc3ccc(N4CCNCC4)cn3)nc2n(C2CCCC2)c1=O]

Palbociclib is described in WHO Drug Information, Vol. 27, No. 2, page 172 (2013). Palbociclib and pharmaceutically acceptable salts thereof are disclosed in International Publication No. WO 2003/062236 and U.S. Pat. Nos. 6,936,612, 7,456,168 and RE47,739; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

The compound, 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (also referred to as “PF-07220060”) is a potent and selective inhibitor of CDK4, having the structure:

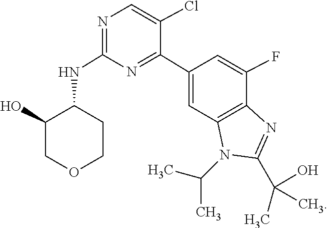 [SMILES: CC(C)n1c(C(C)(C)O)nc2c(F)cc(-c3nc(N[C@@H]4CCOC[C@H]4O)ncc3Cl)cc21]

PF-07220060 and pharmaceutically acceptable salts thereof, are disclosed in International Publication No. WO 2019/207463 published Oct. 31, 2019, U.S. Pat. Nos. 10,766,884 and 11,220,494, and US Patent Publication US 2022/0089580; and International Publication No. WO 2022/058871 published Mar. 24, 2022, the contents of which are incorporated herein by reference in their entirety. Unless indicated otherwise, all references herein to PF-07220060 include references to salts, solvates, hydrates and complexes thereof, and to solvates, hydrates and complexes of salts thereof, including polymorphs, stereoisomers, and isotopically labelled versions thereof.

While CDK4/6 inhibitors have shown significant clinical efficacy in ER-positive metastatic breast cancer, as with other kinases their effects may be limited over time by the development of primary or acquired resistance. The selective CDK4/6 inhibitor palbociclib has proven to be clinically efficacious in breast cancer (DeMichele A, Clark A S, Tan K S, et al. CDK4/6 inhibitor palbociclib (PD-0332991) in Rb+ advanced breast cancer: phase II activity, safety, and predictive biomarker assessment. Clin Cancer Res 2015; 21(5):995-1001; Finn R S, Martin M, Rugo H S, et al. Palbociclib and Letrozole in Advanced Breast Cancer. New Engl J Med 2016; 375(20):1925-36; and Cristofanilli M, Turner N C, Bondarenko I, et al. Fulvestrant plus palbociclib versus fulvestrant plus placebo for treatment of hormone-receptor-positive, HER2-negative metastatic breast cancer that progressed on previous endocrine therapy (PALOMA-3): final analysis of the multicentre, double-blind, phase 3 randomised controlled trial. Lancet Oncol 2016; 17(4):425-39), however, after initial clinical benefit, acquired resistance to palbociclib may occur (Knudsen Erik S., Witkiewicz Agnieszka K., The Strange Case of CDK4/6 Inhibitors: Mechanisms, Resistance, and Combination Strategies. Trends Cancer 2017; 3(1):39-55).

There is a need for appropriate dosing regimens of the oral agent, COMPOUND A as a single agent and in combination therapies for treating cancers, to improve benefit and convenience to patients while minimizing adverse events and risks to patients.

### SUMMARY OF THE INVENTION

The present invention provides, in part, dosing regimens for administering COMPOUND A, or a pharmaceutically acceptable salt thereof, to a subject as a single agent, and in combination therapies, for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention, there is provided a method for treating cancer comprising administering to a subject in need thereof a daily dose of from about 0.1 mg to about 15 mg of a lysine acetyltransferase 6 (KAT6) inhibitor having the structure

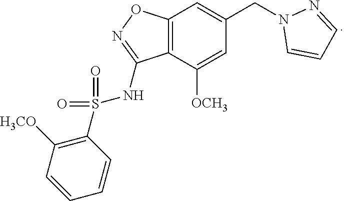 [SMILES: COc1ccccc1S(=O)(=O)Nc1noc2cc(Cn3cccn3)cc(OC)c12]

or a pharmaceutically acceptable salt thereof.

Described below are embodiments of the invention, where for convenience Embodiment 1 (E1) is identical to the embodiment provided above.

It is to be understood that both the foregoing general description and the following detailed description are exemplary and explanatory only and are not restrictive of the invention, as claimed.

### BRIEF DESCRIPTION OF DRAWINGS

FIG. 1 shows the overall study design of an open-label, multi-center, multiple-dose Phase 1 clinical trial in adult patients to evaluate safety, tolerability, pharmacokinetics (PK), and pharmacodynamics (PD) of COMPOUND A in locally advanced or metastatic selected solid tumors and early signs of clinical efficacy of COMPOUND A as a single agent, in combination with an antiestrogen; and in combination with a CDK4 inhibitor and an antiestrogen.

FIG. 2A shows the median COMPOUND A plasma concentration-time profiles on Day 1 following the administration of a single oral dose of COMPOUND A as a single agent or in combination with fulvestrant.

FIG. 2B shows the median COMPOUND A plasma concentration-time profiles on Day 15 following the administration of multiple oral doses of COMPOUND A as a single agent or in combination with fulvestrant.

FIG. 3 shows the steady-state concentration-time profile of COMPOUND A on Day 15 of Cycle 1 of COMPOUND A as a single agent or in combination with fulvestrant. Abbreviations: Fulv=fulvestrant; hr=hour; QD=once daily; and StD=standard deviation.

FIG. 4 shows a waterfall plot for best percentage change from baseline for target lesions by tumor type for a safety analysis set (n=22). Abbreviations: CRPC=castration-resistant prostate cancer; ERBC=ER+/HER2− breast cancer; Fulv=fulvestrant; NSCLC=non-small cell lung cancer; PD=progressive disease; PR=partial response; QD=once daily; and SD=stable disease.

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting.

E1 A method for treating cancer, as defined above.

E2 A method of embodiment E1, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in combination with
a) a cyclin-dependent kinase 4 (CDK4) inhibitor;
b) an antiestrogen; or
c) a CDK4 inhibitor and an antiestrogen.

E3 A method of embodiment E1 or E2, wherein the daily dose of the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered once per day (QD).

E4 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 1 mg to about 15 mg QD.

E5 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 1 mg to about 8 mg QD.

E6 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 0.5 mg to about 5 mg QD.

E7 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 0.1 mg to about 8 mg QD.

E8 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 0.1 mg to less than 1 mg QD.

E9 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 0.1 mg to about 0.75 mg QD.

E10 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of from about 0.5 mg to about 5 mg QD.

E11 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 0.5 mg QD.

E12 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 1 mg QD.

E13 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 2 mg QD.

E14 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 3 mg QD.

E15 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 4 mg QD.

E16 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 5 mg QD.

E17 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 6 mg QD.

E18 A method of any one of embodiments E1 to E3, wherein the 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 7 mg QD.

E19 A method of any one of embodiments E1 to E3, wherein 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in an amount of about 8 mg QD.

E20 A method of any one of embodiments E1 to E19, wherein 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered orally.

E21 A method of any one of embodiments E2 to E20, wherein the CDK4 inhibitor is a CDK4 selective inhibitor or a CDK4/6 inhibitor.

E22 A method of embodiment E21, wherein the CDK4 inhibitor is a CDK4 selective inhibitor.

E23 A method of embodiment E22, wherein the CDK4 selective inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof.

E24 A method of embodiment E21, wherein the CDK4 inhibitor is a CDK4/6 inhibitor.

E25 A method of embodiment E24, wherein the CDK4/6 inhibitor is abemaciclib, ribociclib or palbociclib, or a pharmaceutically acceptable salt thereof.

E26 A method of embodiment E25, the CDK4/6 inhibitor is palbociclib, or a pharmaceutically acceptable salt thereof.

E27 A method of any one of embodiments E2 to E20, wherein the antiestrogen is an aromatase inhibitor, a selective estrogen receptor degrader (SERD) or a selective estrogen receptor modulator (SERM).

E28 A method of embodiment E27, wherein the antiestrogen is fulvestrant or letrozole.

E29 A method of embodiment E28, wherein the antiestrogen is fulvestrant.

E30 A method of embodiment E28, wherein the antiestrogen is letrozole.

E31 A method of any one of embodiments E1 to E30, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

E32 A method of embodiment E31, wherein the cancer is breast cancer, lung cancer, prostate cancer, pancreatic cancer, or ovarian cancer.

E33 A method of embodiment E32, wherein the cancer is breast cancer, lung cancer, or prostate cancer.

E34 A method of embodiment E33, wherein the cancer is breast cancer.

E35 A method of embodiment E34, wherein the breast cancer is hormone receptor positive (HR+) breast cancer.

E36 A method of embodiment E35, wherein the hormone receptor positive (HR+) breast cancer is selected from the group consisting of progesterone receptor positive (PR+) breast cancer and estrogen receptor positive (ER+) breast cancer.

E37 A method of embodiment E36, wherein the breast cancer is progesterone receptor positive (PR+) breast cancer.

E38 A method of embodiment E36, wherein the breast cancer is estrogen receptor positive (ER+) breast cancer.

E39 A method of embodiment E38, wherein the estrogen receptor positive (ER+) breast cancer is human epidermal growth factor receptor 2 negative (HER2−) or the estrogen receptor positive (ER+) breast cancer is human epidermal growth factor receptor 2 positive (HER2+).

E40 A method of embodiment E39, wherein the estrogen receptor positive (ER+) breast cancer is human epidermal growth factor receptor 2 negative (HER2−).

E41 2-methoxy-N-{4-methoxy-6-[(1H-pyrazol-1-yl)methyl]-1,2-benzoxazol-3-yl}benzene-1-sulfonamide, or a pharmaceutically acceptable salt thereof, for use according to any one of embodiments E1 to E40.

E42 Use of 2-methoxy-N-{4-methoxy-6-[(1H-pyrazol-1-yl)methyl]-1,2-benzoxazol-3-yl}benzene-1-sulfonamide, or a pharmaceutically acceptable salt thereof, in the manufacture of a medicament according to any one of embodiments E1 to E41.

Each of the embodiments described herein may be combined with any other embodiment(s) described herein not inconsistent with the embodiment(s) with which it is combined.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

As used herein, the singular form “a”, “an”, and “the” include plural references unless indicated otherwise. For example, “a” substituent includes one or more substituents.

As used herein, the term “about” when used to modify a numerically defined parameter (e.g., the dose of a KAT6 inhibitor) means that the parameter may vary by as much as 10% below or above the stated numerical value for that parameter. For example, a dose of about 5 mg means 5 mg±10%, i.e., it may vary between 4.5 mg and 5.5 mg.

As used herein, terms, including, but not limited to, “agent”, “composition, “compound”, “drug”, and “therapeutic agent” may be used interchangeably to refer to compounds included in the methods and uses of the present invention, specifically a KAT6 inhibitor, a CDK4 inhibitor and an antiestrogen.

As used herein, a “KAT6 inhibitor” includes an inhibitor of KAT6A, an inhibitor of KAT6B, and an inhibitor of KAT6A and KAT6B. KAT6 inhibitors are disclosed in International Publication No. WO2019/043139A1; International Publication No WO2019/243491A1; International Publication No. WO2020/002587; and International Application Serial No. PCT/IB2020/055667. The contents of each of the foregoing references are incorporated herein by reference in their entirety.

Cyclin-dependent kinases (CDKs) and related serine/threonine kinases are important cellular enzymes that perform essential functions in regulating cell division and proliferation. CDK inhibitors include Pan-CDK inhibitors that target a broad spectrum of CDKs or selective CDK inhibitors that target specific CDK(s).

As used herein, a “CDK4 inhibitor” includes a CDK4 selective inhibitor and a CDK4/6 inhibitor. CDK4 selective inhibitors are disclosed in International Publication No. WO 2019/207463. Examples of CDK4/6 inhibitors include, but are not limited to, abemaciclib, ribociclib and palbociclib. Additional examples of CDK4/6 inhibitors include lerociclib (also known as G1T38) and trilaciclib (also known as GTI128).

In an embodiment, CDK4 selective inhibitors of the present invention include 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof.

In an embodiment, a CDK4/6 inhibitor of the present invention includes palbociclib. Unless otherwise indicated herein, palbociclib (also referred to herein as “palbo” or “Palbo”) refers to 6-acetyl-8-cyclopentyl-5-methyl-2-(5-piperazin-1-yl-pyridin-2-ylamino)-8H-pyrido[2,3-d]pyrimidin-7-one, or a pharmaceutically acceptable salt thereof.

As used herein, “endocrine therapy” means an aromatase inhibitor, a selective estrogen receptor degrader (SERD), or a selective estrogen receptor modulator (SERM). In certain embodiments, endocrine therapy includes fulvestrant, tamoxifen, toremifene, anastrozole, exemestane, or letrozole.

The term “antiestrogen” as used herein refers to a class of drugs that prevent estrogens like estradiol from mediating the biological effects in the body. Antiestrogens act by blocking the estrogen receptor (ER) and/or inhibiting or suppressing estrogen production. In other embodiments, an antiestrogen is an aromatase inhibitor, a selective estrogen receptor degrader (SERD) or a selective estrogen receptor modulator (SERM). Examples of an aromatase inhibitor include, but are not limited to, anastrozole. Examples of a SERD include, but are not limited to, fulvestrant. Additional SERDs include elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). Examples of a SERM include, but are not limited to, tamoxifen, clomifene and raloxifene. Additional SERMS include toremifene, lasofoxifene, bazedoxifene and afimoxifene.

In an embodiment, the aromatase inhibitor includes letrozole, exemestane, and anastrozole. In an embodiment, the SERM includes tamoxifen, clomifene and raloxifene.

In an embodiment, an antiestrogen of the present invention includes fulvestrant and letrozole. In an embodiment, an antiestrogen of the present invention includes fulvestrant. In an embodiment, an antiestrogen of the present invention includes letrozole.

Another embodiment relates to the pharmaceutically acceptable salts of the compounds described herein. Pharmaceutically acceptable salts of the compounds described herein include the acid addition and base addition salts thereof.

Another embodiment also relates to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

### Administration and Dosing

“Treat” or “treating” a cancer and/or a cancer-associated disease as used herein means to administer a monotherapy or combination therapy according to the present invention to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment” or “therapy,” as used herein, unless otherwise indicated, refers to the act of treating as “treating” is defined immediately above. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (2009)).

As used herein, the terms, “subject”, “participant” and “patient,” are used interchangeably, to a human. Human subjects may be of any gender. In an embodiment, a human is an adult human.

An “amount” for use and for treating a subject refers to an amount that provides, in single or multiple doses, alone, or in combination with one or more other agents, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent, alone, or in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

Embodiments of the present invention provide a dose, dosage and dosing regimen comprising administering to a subject an amount, or a therapeutically effective amount, of COMPOUND A or a pharmaceutically acceptable salt thereof. The amount, or the therapeutically effective amount, can be a daily dose in the range of from about 0.1 mg to about 15 mg. In another embodiment, a daily dose is from about 1 mg to about 15 mg, a daily dose is from about 1 mg to about 10 mg, from about 1 mg to about 8 mg, a daily dose is from about 0.1 mg to about 8 mg, from about 1 mg to about 5 mg, from about 0.1 mg to about 5 mg, or from about 0.5 mg to about 5 mg. In another embodiment, a daily dose is from about 0.1 mg to less than 1 mg or from about 0.1 mg to about 0.75 mg. In preferred embodiments, the daily dose is about 0.5 mg, 1 mg, about 2 mg, about 3 mg, about 4 mg, about 5 mg, about 6 mg, about 7 mg or about 8 mg. In preferred embodiments, the daily dose is about 0.5 mg, about 1 mg, about 2 mg, about 3 mg, about 4 mg, or about 5 mg. In preferred embodiments, the daily dose is 0.5 mg, 1 mg, 2 mg, 3 mg, 4 mg, 5 mg, 6 mg, 7 mg or 8 mg. In preferred embodiments, the daily dose is 0.5 mg, 1 mg, 2 mg, 3 mg, 4 mg, or 5 mg.

In another embodiment, COMPOUND A or a pharmaceutically acceptable salt thereof, may be administered, either as a single agent or in combination with an antiestrogen, in an amount sufficient to yield a maximum plasma concentration (Cₘₐₓ) at steady state in the subject of from 400 to 13000 ng/mL, for example, from 500 to 700 ng/mL, from 1400 to 2800 ng/mL, from 2000 to 4400 ng/mL, or from 4000 to 12000 ng/mL, for example, after daily 2 mg, 5 mg, 8 mg, or 15 mg oral administration of COMPOUND A.

In another embodiment, COMPOUND A or a pharmaceutically acceptable salt thereof, may be administered, either as a single agent or in combination with an antiestrogen, in an amount that provides a maximum plasma concentration (Cₘₐₓ) at steady state in the subject of from 400 to 13000 ng/mL, for example, from 500 to 700 ng/mL, from 1400 to 2800 ng/mL, from 2000 to 4400 ng/mL, or from 4000 to 12000 ng/mL. In an embodiment thereof, the COMPOUND A is administered at a daily dose of from about 1 mg to about 15 mg. In an embodiment thereof, the COMPOUND A is administered at a daily dose of 1 mg, 2 mg, 3 mg, 4 mg, 5 mg, 6 mg, 7 mg or 8 mg.

In a preferred embodiment, the daily dose of COMPOUND A or a pharmaceutically acceptable salt thereof, is administered once per day (QD).

The compounds of the invention may be administered orally. Oral administration may involve swallowing, so that the compound enters the gastrointestinal tract, or buccal or sublingual administration may be employed by which the compound enters the bloodstream directly from the mouth.

In a preferred embodiment, the daily dose of COMPOUND A or a pharmaceutically acceptable salt thereof, is administered orally.

COMPOUND A, or a pharmaceutically acceptable salt, may be present in a pharmaceutical composition which includes a pharmaceutically acceptable excipient. “Pharmaceutically acceptable excipient” refers to a component that may be included in the compositions described herein, is physiologically suitable for pharmaceutical use, and causes no significant adverse effects nor therapeutic effects to a subject. The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

The amount of COMPOUND A, or a pharmaceutically acceptable salt, in the pharmaceutical compositions can be any amounts disclosed herein.

The compounds of the method, use or combination of the present invention may be formulated prior to administration. The formulation will preferably be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous and nonaqueous oral solutions and suspensions, packaged in containers adapted for subdivision into individual doses.

In another embodiment, the dosage of a compound or pharmaceutical composition described herein can vary within the range depending upon the dosage form employed. In another embodiment, an amount of a compound or pharmaceutical composition described herein administered to a subject can be dependent upon factors known to a skilled artisan. Further, it will be understood that the specific dose of a pharmaceutical composition comprising a compound as disclosed herein can depend on a variety of factors including physical condition of the subject (e.g., age, gender, weight), and medical history of the subject (e.g., medications being taken, health condition other diseases or disorders).

In an embodiment, palbociclib, or a pharmaceutically acceptable salt thereof, is administered at a daily dosage of about 125 mg once daily, about 100 mg once daily, about 75 mg once daily, about 50 mg daily, or about 25 mg daily. In an embodiment, which is the recommended starting dose, palbociclib, or a pharmaceutically acceptable salt thereof, is administered at a daily dosage of about 125 mg once a day. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered at a dose of about 100 mg once daily. In an embodiment, palbociclib, or a pharmaceutically acceptable salt thereof, is administered at a dose of about 75 mg once daily. In an embodiment, palbociclib, or a pharmaceutically acceptable salt thereof, is administered at a dose of about 50 mg once daily. Dosage amounts provided herein refer to the dose of the free base form of palbociclib, or are calculated as the free base equivalent of an administered palbociclib salt form. For example, a dosage or amount of palbociclib, such as 100 mg, 75 mg or 50 mg, refers to the free base equivalent.

In an embodiment, 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof, is administered at a daily dosage of from about 1 mg to about 1000 mg per day. In another embodiment, the CDK4 inhibitor is administered at a daily dosage from about 10 mg to about 500 mg per day. In another embodiment, the CDK4 inhibitor is administered at a dosage of from about 25 mg to about 300 mg per day, preferably on a BID schedule. In another embodiment, the CDK4 inhibitor is administered at a dosage of from about 100 mg to about 300 mg on a BID schedule. In another embodiment, the CDK4 inhibitor is administered at a dosage of about 100 mg on a BID schedule. In another embodiment, the CDK4 inhibitor is administered at a dosage of about 300 mg on a BID schedule. In another embodiment the CDK4 inhibitor is administered at dosages of about: 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, BID, TID or QID schedule.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In an embodiment, 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered once daily to comprise a complete cycle of 28 days. Repetition of the 28 day cycles is continued during treatment with the combination of the present invention.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28 days. Repetition of the 28 day cycles is continued during treatment with the combination of the present invention.

The standard clinical dosing regimen, for palbociclib, or a pharmaceutically acceptable salt thereof, is administration of 125 mg once daily for 21 consecutive days followed by 7 days off treatment to comprise a complete cycle of 28 days. Repetition of the 28 day cycles is continued during treatment with the combination of the present invention.

In further embodiments of the invention, 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in combination with palbociclib and letrozole, where the palbociclib is administered at 125 mg orally, once daily for 21 days followed by 7 days off, and where the letrozole is administered at 2.5 mg orally, daily.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

### Method of Treatment

In one embodiment, the disclosure provides a method for treating cancer of a subject in need thereof, which includes administering to the subject an amount of COMPOUND A as described herein. In another embodiment, the disclosure also provides a method for treating cancer of a subject which includes administering to the subject an amount of COMPOUND A as described herein in combination with a) an amount of a cyclin-dependent kinase 4 (CDK4) inhibitor; b) an amount of an antiestrogen; or c) an amount of a CDK4 inhibitor and an amount of an antiestrogen.

The term “combination”, as used herein, unless otherwise indicated, means a fixed-dose combination or a combination of agents that is administered intermittently, concurrently or sequentially, according to the same or different route of administration and according to the same or different dosage schedules. As used herein, an “effective” or a “therapeutically effective” amount refers to an amount of an agent, compound, or composition that is of sufficient quantity to result in a decrease in severity of disease symptoms, an increase in frequency and duration of disease symptom-free periods, or a prevention of impairment or disability due to the disease affliction—either as a single dose or according to a multiple dose regimen, alone or in combination with other agents. One of ordinary skill in the art would be able to determine such amounts based on such factors as the patient's size, the severity of the patient's symptoms, and the particular combination, composition or route of administration selected. The patient or subject may be a human or non-human mammal in need of treatment. In one embodiment, the patient is human.

The term “locally advanced”, as used herein, as it relates to cancer, may or may not be treated with curative intent. The term “metastatic” as used herein, as it relates to cancer, cannot be treated with curative intent. Those skilled in the art will be able to recognize and diagnose locally advanced and metastatic cancer in a patient.

For convenience, certain well-known abbreviations, may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC) and progesterone receptor (PR). The abbreviations, ER+HER2−, ER+HER2−, and ER+/HER2−, as they relate to a breast cancer indication are equivalent and exchangeable.

In one embodiment, the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, or a combination of two or more of the foregoing cancers.

Another embodiment relates to methods of treating cancer in a patient. Another embodiment relates to the treatment of cancer in a patient comprising administering to the patient an amount of the compounds described herein that are effective in treating the cancer.

In one embodiment, the cancer is breast, lung, colon, brain, head and neck, prostate, stomach, pancreatic, ovarian, melanoma, endocrine, uterine, testicular, or bladder.

In one embodiment, the cancer is breast, lung, prostate, pancreatic, or ovarian.

In one embodiment, the cancer is breast, lung, or prostate.

In one embodiment, the cancer is breast cancer.

In one embodiment, the breast cancer is HR+ breast cancer.

In one embodiment, the HR+ breast cancer is PR+ and/or ER+ breast cancer.

In one embodiment, the breast cancer is PR+ breast cancer.

In one embodiment, the breast cancer is ER+ breast cancer.

In one embodiment, the breast cancer is ER+HER2− breast cancer.

In one embodiment, the breast cancer is ER+HER2+ breast cancer.

In one embodiment, the breast cancer is locally advanced or metastatic ER+ breast cancer.

In one embodiment, the breast cancer is locally advanced or metastatic ER+HER2− breast cancer.

In one embodiment, the breast cancer is locally advanced or metastatic ER+HER2+ breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer.

In one embodiment, the prostate cancer is locally advanced or metastatic castration resistant prostate cancer.

Another embodiment relates to methods of treating solid tumors in a patient. Another embodiment relates to the treatment of solid tumors in a patient comprising administering to the patient an amount of the compounds described herein that are effective in treating the solid tumor.

In one embodiment, the solid tumor is breast, lung, colon, brain, head and neck, prostate, stomach, pancreatic, ovarian, melanoma, endocrine, uterine, testicular, or bladder.

In one embodiment, the solid tumor is breast, lung, prostate, pancreatic, or ovarian.

In one embodiment, the solid tumor is breast, lung, or prostate.

In one embodiment, the solid tumor is breast cancer, and in a further embodiment, the breast cancer is HR+ breast cancer, and in a still further embodiment the HR+ breast cancer is PR+ and/or ER+ breast cancer.

In one embodiment, the solid tumor is breast cancer, and in a further embodiment, the breast cancer is ER+HER2− breast cancer.

In one embodiment, the solid tumor is breast cancer, and in a further embodiment, the breast cancer is ER+HER2+ breast cancer.

In one embodiment, the solid tumor is breast cancer, and in a further embodiment, the breast cancer is locally advanced or metastatic ER+HER2− breast cancer.

In one embodiment, the solid tumor is breast cancer, and in a further embodiment, the breast cancer is locally advanced or metastatic ER+HER2+ breast cancer.

In one embodiment, the solid tumor is lung cancer, and in a further embodiment the lung cancer is non-small cell lung cancer.

In one embodiment, the solid tumor is lung cancer, and in a further embodiment the lung cancer is locally advanced or metastatic non-small cell lung cancer.

In one embodiment, the solid tumor is prostate cancer, and in a further embodiment the prostate cancer is castration resistant prostate cancer.

In one embodiment, the solid tumor is prostate cancer, and in a further embodiment the prostate cancer is locally advanced or metastatic castration resistant prostate cancer.

Another embodiment relates to methods of treating hematologic tumors in a patient.

Another embodiment relates to the treatment of hematologic tumors in a patient comprising administering to the patient an amount of the compounds described herein that is effective in treating the hematologic tumor.

In one embodiment, the hematologic tumor is leukemia, lymphoma or multiple myeloma.

In one embodiment, the hematologic tumor is leukemia or lymphoma.

Another embodiment relates to methods of treating cancer in a patient with locally advanced or metastatic ER+HER2− breast cancer, CRPC, or NSCLC whose disease progressed on or is intolerant to standard therapy.

Another embodiment relates to methods of treating cancer in a patient with locally advanced or metastatic ER+HER2− breast cancer, CRPC, or NSCLC whose disease progressed on or is intolerant to standard therapy.

Another embodiment relates to methods of treating cancer in a patient with locally advanced or metastatic 2L+ER+HER2 breast cancer who has progressed after at least 1 prior line of treatment with an endocrine therapy and CDK4/6 inhibitor. In an embodiment thereof, the patient is administered a combination of COMPOUND A and fulvestrant.

Another embodiment relates to methods of treating cancer in a patient with locally advanced or metastatic 2L+ER+HER2 breast cancer who has progressed after at least 1 prior line of treatment with an endocrine therapy and CDK4/6 inhibitor. In an embodiment thereof, the patient is administered a combination of COMPOUND A with letrozole and palbociclib.

Another embodiment relates to methods of treating cancer in a patient with advanced or metastatic 2L+ER+HER2− breast cancer who has progressed after at least 1 prior line of CDK4/6 inhibitor and 1 line of endocrine therapy. In an embodiment thereof, the patient is administered a COMPOUND A.

Another embodiment relates to methods of treating cancer in a patient with advanced or metastatic 2-4L fulvestrant-naïve ER+HER2− breast cancer whose disease has progressed after 1 line of a CDK4/6 inhibitor and 1 line of endocrine therapy and who must not have received more than 3 lines of systemic therapies in advanced or metastatic setting. In an embodiment thereof, the patient is administered a COMPOUND A and fulvestrant.

### EXAMPLES

In order that this invention may be better understood, the following examples are set forth. These examples are for purposes of illustration only and are not to be construed as limiting the scope of the invention in any manner.

### Phase 1 Compound A Clinical Trial

### Overview

COMPOUND A is being investigated in an ongoing open-label, multi-center, multiple-dose Phase 1 study in adult patients to evaluate safety, tolerability, PK, and PD of COMPOUND A in locally advanced or metastatic selected solid tumors (ER+HER2− breast cancer, CRPC, or NSCLC) and early signs of clinical efficacy of COMPOUND A as a single in combination with an antiestrogen; and in combination with a CDK4 inhibitor and an antiestrogen. The patients in this trial are intolerant of or resistant to standard therapy.

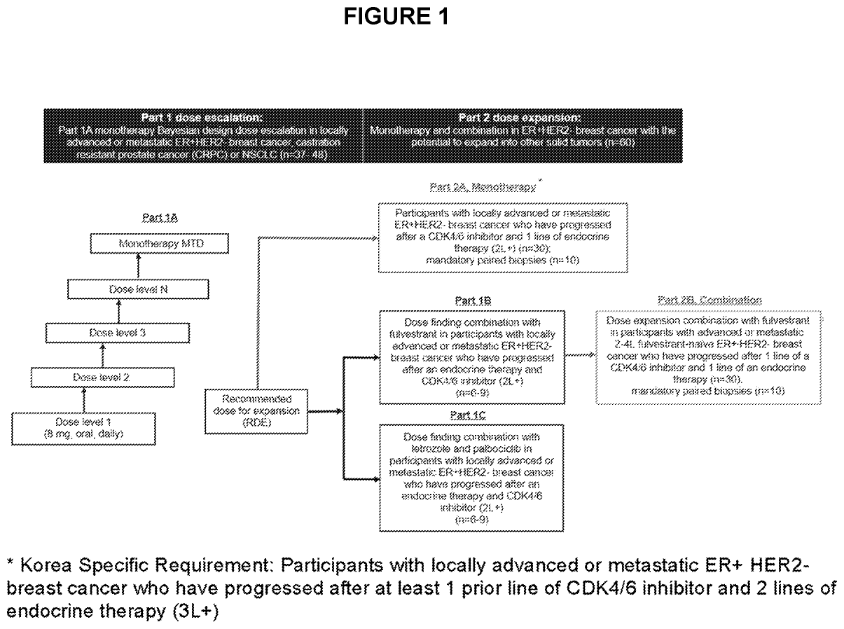

Study Design: The overall study design is depicted in FIG. 1. The study contains two parts, a dose escalation (Part 1) followed by a dose expansion (Part 2). Not shown in FIG. 1 are Part 1D and Part 2D, which are additional arms in Part 1 and Part 2 in the study design and are described below.

As of the data cut-off date of 23 Mar. 2022, 31 participants were treated with COMPOUND A and early signs of clinical efficacy of COMPOUND A were observed.

### Example 1: Pharmacokinetics (PK) Study—Data Cut-Off Date of 23 Mar. 2022

COMPOUND A was orally administered at 2 mg, 5 mg, 8 mg, and 15 mg QD alone or 5 mg QD in combination with 500 mg of fulvestrant.

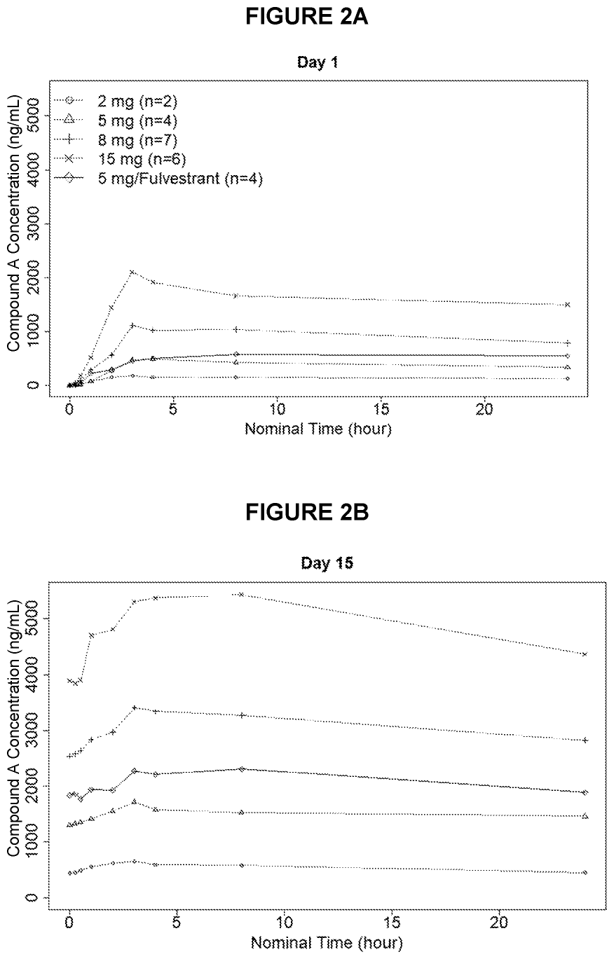

FIG. 2A shows the median COMPOUND A plasma concentration versus time profiles on Day 1 following the administration of a single oral dose of COMPOUND A at 2 mg, 5 mg, 8 mg, and 15 mg QD alone and 5 mg QD in combination with 500 mg of fulvestrant. FIG. 2B shows the median COMPOUND A plasma concentration versus time profiles on Day 15 following the administration of multiple oral doses of COMPOUND A at 2 mg, 5 mg, 8 mg, and 15 mg QD alone and 5 mg QD in combination with 500 mg of fulvestrant.

Preliminary pharmacokinetic parameters are available from 23 participants following the 1^{st} oral dose (Day 1 of Cycle 1) and at steady state (Day 15 of Cycle 1) and presented in Table 1.

TABLE 1
Preliminary Plasma Summary of COMPOUND A Pharmacokinetic Parameters after
1st dose (Day 1 of Cycle 1) and at steady state (Day 15 of Cycle 1)

| | | Monotherapy | | | | Combination |
|---|---|---|---|---|---|---|
| | | Geometric mean (CV %) * | | | | with fulvestrant |
| | | Dose (mg) | | | | |
| | PK parameters | 2 | 5 | 8 | 15 | 5 |
| | N | 2 | 4 | 7 | 6 | 4 |
| C1D1 | Cₘₐₓ (ng/mL) | 133, 224 | 508 (16.5) | 1051 (24.4) | 1936 (32.6) | 641 (16.3) |
| | Tₘₐₓ (hr)** | 2, 3 | 3.5 (2, 4) | 3 (2, 8) | 3.5 (2, 4) | 6 (3, 24) |
| | AUC₂₄ (μg*hr/mL) | 2.5, 3.9 | 8.9 (6.2) | 20.5 (23.4) | 35.7 (29.6) | 12.1 (16.4) |
| C1D15 | Cₘₐₓ (ng/mL) | 583, 724 | 1709 (18.1) | 3620 (12.6) | 6028 (43.2) | 2344 (20.4) |
| | Tₘₐₓ (hr)** | 3, 3 | 3 (3, 8) | 3 (2, 8) | 3 (3, 8) | 6 (3, 8) |
| | AUC₂₄ (μg*hr/mL) | 11.2, 14.3 | 35.9 (23.9) | 76.5 (9.7) | 127.1 (43.2) | 50.1 (26.9) |
| | Rac** | 2.9, 5.8 | 4.1 (2.9, 5.5) | 3.8 (3.1, 5.1) | 3.8 (2.3, 4.7) | 4.2 (3.2, 5.1) |
| | CL/F (L/hr) | 0.14, 0.18 | 0.14 (23.9) | 0.10 (9.7) | 0.12 (43.2) | 0.10 (26.9) |

* only minimum and maximum are presented for 2 mg;
**median (minimum, maximum).
Abbreviations: C1D1: Cycle 1 Day 1; C1D15: Cycle 1 Day 15; CV: coefficient of variance; N: number of patients; Cmax: maximal concentration after the dose; Tₘₐₓ: time when maximal concentration was achieved; AUC₂₄: area under the curve from time 0 to 24 hours after the dose; CL/F: apparent clearance of COMPOUND A; Rac: ratio of AUC₂₄ at Cycle 1 Day 15 to AUC₂₄ at Cycle 1 Day 1

Following repeated daily dosing to Day 15, COMPOUND A was absorbed with a median Tₘₐₓ of 3 hours. COMPOUND A accumulated following repeated daily oral dosing with Rac ranged from 2.3 to 5.8. From a dose range of 2 mg to 15 mg administered alone or in combination with fulvestrant, COMPOUND A exposure, i.e. AUC₂₄, increased proportionally when dose was increased. The apparent clearance, CL/F, at each dose level, are similar, suggesting that the pharmacokinetic of COMPOUND A was linear and no apparent drug-drug interaction between COMPOUND A and fulvestrant. The inter-patient variability of the drug is low to moderate, i.e. CV % for AUC₂₄ at Day 1S of Cycle 1 ranged from 9.7% to 43.2% and CV % for Cₘₐₓ at Day 1S of Cycle 1 ranged from 12.6% to 43.2%.

### Example 1A: Pharmacokinetics (PK) Study—Data Cut-Off Date of 30 Sep. 2022

1S COMPOUND A was orally administered at 1 mg, 2 mg, 5 mg, 8 mg, and 15 mg QD alone or 5 mg QD in combination with 500 mg of fulvestrant.

A total of 29 participants had evaluable PK concentration data in Part 1A and Part 1B, at the time of the data cut-off date of 30 Sep. 2022. Of these participants, two (2) in the 1 mg QD dose group were excluded from Cycle 1 Day 15 summaries due to either dose interruption impacting PK analysis or sample collection after the data cut-off date.

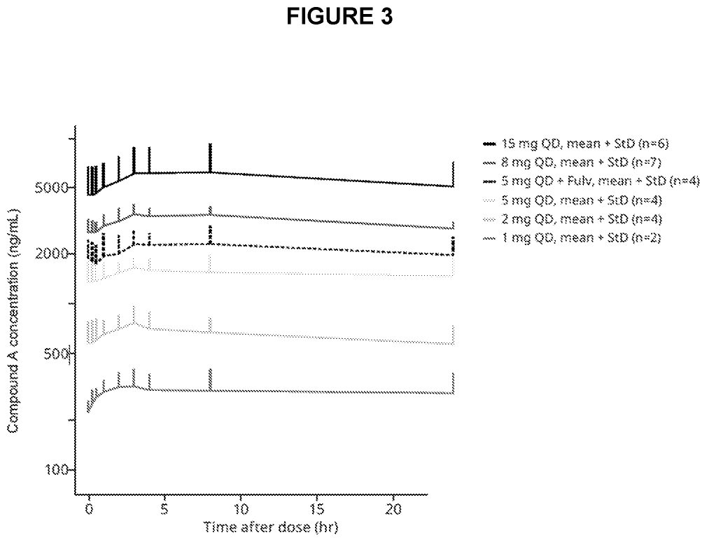

FIG. 3 shows the steady-state concentration-time profile of COMPOUND A on Day 15 of Cycle 1. Compound A PK was linear between 1 mg and 15 mg QD dose regimens and steady state was achieved by Cycle 1 Day 15, as shown in FIG. 3. The steady-state concentrations were near or above Cₑrf targets (defined from preclinical models) when dosed at ≥1 mg.

### Example 2: Safety and Efficacy—Data Cut-Off Date of 23 Mar. 2022

As of the data cut-off 23 Mar. 2022, safety and efficacy of COMPOUND A is being assessed in an ongoing, first-in-human, Phase 1 Study. Overall, 31 participants were treated with COMPOUND A. Dose de-escalation with monotherapy is ongoing (Part 1A); Recommended dose for expansion (RDE) for monotherapy was identified as 5 mg QD and Part 1B is ongoing. RDE for combination with fulvestrant was also identified as 5 mg QD and dose expansion is ongoing (Part 2B).

### 1. Study Design

### Part 1 (Dose Escalation)

Part 1 dose escalation further divides into Part 1A, Part 1B, Part 1C, and Part 1D.

Part 1A (Monotherapy Dose Escalation) contains dose escalation as monotherapy in patients with locally advanced or metastatic ER+HER2− breast cancer, CRPC, or NSCLC that is resistant or intolerant to standard therapy or for whom no standard therapy is available, to determine the maximum tolerated dose (MTD) and select the RDE. Participants received escalating doses of COMPOUND A starting from 8 mg QD orally. A 2-parameter Bayesian Logistic Regression Model (BLRM) was used for dose finding.

Part 1B (Combination Dose Escalation), COMPOUND A in combination with fulvestrant was evaluated for dose finding in patients with locally advanced or metastatic ER+HER2− breast cancer (2L+) who progressed after at least one line of treatment with an endocrine therapy and a CDK4/6 inhibitor to determine the MTD and RDE for this combination. Combination RDE may be different from monotherapy RDE due to potential toxicity overlap. A 5-parameter BLRM was used for dose finding.

Part 1C (Combination Dose Escalation), COMPOUND A in combination with letrozole+palbociclib evaluates dose finding in patients with locally advanced or metastatic ER+HER2− breast cancer (2L+) who progressed after at least one line of treatment with an endocrine therapy and a CDK4/6 inhibitor to determine the MTD and RDE for this combination.

Part 1D (Combination Dose Escalation), COMPOUND A in combination with fulvestrant+PF-07220060 evaluates dose finding in patients with locally advanced or metastatic ER+HER2− breast cancer (2L+) who progressed after at least one line of treatment with an endocrine therapy and a CDK4/6 inhibitor to determine the MTD and RDE for this combination.

BLRM specifically developed for double and triple combinations is used for dose finding in Part 1B, Part 1C and Part 1D.

### Part 2 (Dose Expansion)

Part 2A (ER+HER2− breast cancer 2L+, monotherapy): After selection of the monotherapy RDE in Part 1A, patients having locally advanced or metastatic ER+HER2− breast cancer (2L+) who progressed after at least 1 prior line of CDK4/6 inhibitor and 1 line of endocrine therapy are evaluated in a dose-expansion cohort using COMPOUND A as a monotherapy.

Part 2B (ER+HER2− breast cancer 2-4L, fulvestrant-naïve, combination with fulvestrant): After determination of the combination RDE from Part 1B, patients with advanced or metastatic 2-4L fulvestrant-naïve ER+HER2− breast cancer whose disease progressed after 1 line of a CDK4/6 inhibitor and 1 line of endocrine therapy and who have not received more than 3 lines of systemic therapies in advanced or metastatic setting are evaluated in a dose-expansion combination cohort using COMPOUND A in combination with fulvestrant.

Part 2D (ER+HER2− breast cancer 2-4L, combination with PF-07220060 and fulvestrant): After determination of the combination RDE from Part 1D, patients with advanced or metastatic ER+HER2− breast cancer whose disease progressed after 1 line of a CDK4/6 inhibitor and 1 line of endocrine therapy and who have not received more than 3 lines of systemic therapies including up to 1 line of cytotoxic chemotherapy for visceral disease in advanced or metastatic setting are evaluated in a dose-expansion combination cohort using COMPOUND A in combination with fulvestrant+PF-07220060.

As of 23 Mar. 2022, 31 participants were treated with COMPOUND A in dose escalation (Part 1) and dose expansion (Part 2).

### Method of Administration

Based on preclinical data, COMPOUND A is predicted to exhibit a low plasma CL of ˜0.1 mL/min/kg and low Vₛₛ of ˜0.1 L/kg, resulting in t of approximately 12 h, which is suitable for QD dosing in humans with high oral bioavailability.

COMPOUND A was orally administered at escalating doses of 2, 5, 8, and 15 mg QD alone or at 5 mg QD in combination with fulvestrant. COMPOUND A is being orally administered at 1 mg QD alone or at 5 mg QD in combination with fulvestrant. COMPOUND A was administered orally at doses of 0.5 mg, 1 mg, 2 mg, and 5 mg QD in combination with a fixed dose of fulvestrant and varying doses of PF-07220060. COMPOUND A may start at 1 dose level below the monotherapy RDE (RDE-1) with fixed doses of fulvestrant or letrozole+palbociclib, and varying doses of PF-07220060.

In addition, depending on the safety findings in Part 1A, the starting dose of COMPOUND A in combination may be further modified to a lower dose.

Participants were to swallow COMPOUND A whole tablets and were not to manipulate or chew the study intervention prior to swallowing. COMPOUND A was administered QD by mouth for all cohorts on a continuous basis. The once daily dose was administered in 24±3 hour intervals (i.e., no less than 21 hours and no more than 27 hours apart). All cycles were 28 days in length.

Fulvestrant 500 mg was administered intramuscularly into the buttocks slowly (1-2 minutes per injection) as two 5 mL injections, one in each buttock, and once monthly thereafter according to product labeling and in compliance with its local prescribing information.

Letrozole was administered orally at 2.5 mg once daily (QD) as continuous daily dosing schedule according to product labeling and in compliance with its local prescribing information.

Palbociclib was administered orally once a day at 125 mg/day for 21 days followed by 7 days off treatment for each 28-day cycle according to product labeling and in compliance with its local prescribing information.

PF-07220060 was administered orally at 100 mg or 300 mg twice daily (BID).

Treatment continues until progression of disease, uncontrollable toxicity, a decision by the patient or investigator to discontinue treatment or the study is terminated.

Patients experiencing toxicity including a dose limiting toxicity (DLT) are managed with dose modification or discontinuation from treatment.

### Definitions

As used herein, “dose limiting toxicity” (DLT) refers to the dosage of COMPOUND A that is contraindicative of a further increase in dosage. For monotherapy dose escalation (Part 1A) and combination dose escalation (Part 1B and Part 1C), any of the following adverse events (AEs) occurring in the first cycle of treatment (28 days), which are attributable to COMPOUND A or any combination therapy agent, as applicable, were classified as DLTs:

### Hematological Dose-Limiting Toxicities:

Any Grade ≥4 hematologic possibly treatment-related AE is a DLT with the following clarifications:
Grade 4 neutropenia regardless of intervention is a DLT.
Febrile neutropenia (defined as an absolute neutrophil count (ANC)<1000/mm3 with a single temperature of >38.3° C. [101° F.], or a sustained temperature of ≥38° C. [100.4° F.] for more than 1 hour) is a DLT.
Grade 3 neutropenia with infection is a DLT.
Grade 3 neutropenia lasting >7 days is a DLT.
Grade 4 thrombocytopenia is a DLT.
Grade 3 thrombocytopenia with bleeding or requiring platelet transfusion is a DLT.
Grade 4 anemia is a DLT.
Grade 3 anemia requiring blood transfusion is a DLT.

### Non-Hematologic Dose-Limiting Toxicities:

Any Grade ≥3 non-hematologic possibly treatment related AE is a DLTs with the following clarifications:
Grade ≥3 nausea, vomiting, or diarrhea lasting 3 days despite adequate antiemetic and other supportive care is a DLT.
Grade ≥3 fatigue lasting ≥5 days is a DLT.
Confirmed drug-induced liver injury (DILI) meeting Hy's law criteria is a DLT.
For participants with Grade 2 hepatic transaminase or alkaline phosphatase levels at baseline as a result of liver metastasis or bone metastasis, aspartate aminotransferase (AST) or alanine aminotransferase (ALT)>8×upper limit of normal (ULN) or AST or ALT>5×ULN for ≥14 days will be considered as a DLT.
Clinically important or persistent toxicities (e.g., toxicities responsible for significant dose delay) that are not included in the above criteria may also be considered a DLT following review by the investigators and the Sponsor. All DLTs need to represent a clinically significant shift from baseline.
Grade ≥3 QTc prolongation is a DLT.
Grade ≥3 anaphylaxis is a DLT.

Any toxicity causing greater than 2 weeks of dose delay is a DLT. In addition, any Grade 5 AE (death) not clearly due to either the underlying disease or other etiologies is a DLT.

Any dose reduction due to a treatment-related AE (per protocol) during the first 28 days will be qualified as participant experiencing DLT.

As used herein “maximum tolerated dose” (MTD) refers to the highest dosage of COMPOUND A that does not cause unacceptable side effects or intolerable toxicities. MTD is defined as a dose with true DLT rate from the target toxicity interval. The target interval for the DLT rate is defined as (0.16, 0.33).

### II. Safety

### Dose Limiting Toxicities (DLTs)

A patient was classified as DLT-evaluable if the participant experiences a DLT or received >75% of the planned doses and had received all scheduled safety assessments during the DLT window. As of 23 Mar. 2022, 19 patients were treated in monotherapy dose escalation, 6 participants at 15 mg QD, 7 patients at 8 mg QD, 4 patients at 5 mg QD, and 2 participants at 2 mg QD. Four participants were treated in combination dose escalation at 5 mg QD+fulvestrant. Three dose limiting toxicities (DLTs) were reported during the study. Of these, 1 DLT was reported at 2 mg QD (monotherapy escalation), 1 DLT was reported at 8 mg QD (monotherapy escalation), and 1 DLT was reported at 5 mg QD+fulvestrant (combination escalation).

All 3 DLTs were of Grade 3 neutropenia (decreased neutrophil count).

### Adverse Events

Adverse events (AEs) of the patients dosed with COMPOUND A in the Phase 1 clinical trial were coded according to the medical dictionary for regulatory activities (MedDRA), version 24.1. The severity of adverse events were graded according to the National Cancer Institute Common Terminology Criteria for Adverse Events (NCI CTCAE) version 5.0.

Treatment-Emergent all-Causality Adverse Events

As of 23 Mar. 2022, 31 patients dosed with COMPOUND A experienced at least one treatment emergent adverse event (TEAE). The most common TEAEs occurring in ≥20% of patients was Dysgeusia (N=26, 83.9%); Anemia (N=17, 54.8%); Neutrophil count decreased/Neutropenia (N=16, 51.6%); Diarrhea (N=11, 35.5); Aspartate aminotransferase increased and White blood cell count decreased (N=9, 29.0% each); Alanine aminotransferase increased and Fatigue (N=8, 25.8%); and Platelet count decreased in (N=7, 22.6%).

All-causality Grade 3 TEAEs included Neutrophil count decreased/Neutropenia (N=9, 29.1%); Anemia (N=5, 16.1%); White blood cell count decreased (N=3, 9.7%); Alanine aminotransferase increased, Lymphocyte count decreased and Hypotension (N=2, 6.5%); Diarrhea, Fatigue, Thrombocytopenia, Urinary tract infection, Haematuria, Anaphylactic reaction, COVID-19, Embolism, Flank pain and Pulmonary embolism (N=1, 3.2%).

One Grade 4 (Hypercalcaemia) and one Grade 5 (Pneumonitis) (N=1, 3.2%) TEAEs were reported.

### Treatment-Emergent Treatment Related Adverse Events

As of 23 Mar. 2022, 31 patients dosed with COMPOUND A experienced at least one treatment-related adverse event (TRAE). The most common TRAEs occurring in 20% of patients was Dysgeusia (N=25, 80.6%); Anemia (N=17, 54.8%); Neutrophil count decreased/Neutropenia (N=16, 51.6%); Diarrhea and White blood cell count decreased (N=9, 29.0%); and Platelet count decreased (N=7, 22.6%).

Grade 3 treatment-related AEs include Neutrophil count decreased/Neutropenia (N=9, 29.1%); Anemia (N=5, 16.1%); White blood cell count decreased (N=3, 9.7%); Diarrhea, Lymphocyte count decreased, Thrombocytopenia and Embolism (N=1, 3.2%).

One Grade 5 treatment-related AE, Pneumonitis (N=1, 3.2%) was reported. There were no Grade 4 treatment-related AEs.

### All-Causality and Treatment Related Serious Adverse Events (SAEs)

A total of ten (10) all-causality SAEs were reported in eight (8) patients as of 23 Mar. 2022 (Table 2). Two (2) Treatment-related SAEs Pneumonitis and Hypotension were reported (Table 3).

TABLE 2
Summary of Serious Adverse Events -
All-Causality by Patient Frequency

| Preferred Event Term | Total |
|---|---|
| COVID-19 | 2 |
| Hypotension | 2 |
| Bone pain | 1 |
| Dysphagia | 1 |
| Embolism | 1 |
| Flank pain | 1 |
| Haematuria | 1 |
| Pneumonitis | 1 |
| Total Number of Cases | 9 |
| Total Number of events | 10 |
| Total Number of Subjects | 8 |

TABLE 3
Summary of Serious Adverse Events - Treatment-
Related by Patient Frequency

| Preferred Event Term | Total |
|---|---|
| Pneumonitis | 1 |
| Hypotension | 1 |
| Total Number of Cases | 2 |
| Total Number of events | 2 |
| Total Number of Subjects | 2 |

### III. Efficacy

As of 23 Mar. 2022, signs of efficacy in ER+HER2− breast cancer patients were observed during dose escalation. Additional analysis is ongoing in dose expansion.

### IV. Patient Population

Demographic characteristics for patients treated with COMPOUND A in the Phase I Study are shown in Tables 4 and 5.

TABLE 4
Demographic Characteristics for Phase I Study

| | | | | COMPD A |
|---|---|---|---|---|
| COMPD A | COMPD A | COMPD A | COMPD A | Total PART |
| 2 mg QD | 5 mg QD | 8 mg QD | 15 mg QD | 1A |
| (Monotherapy | (Monotherapy | (Monotherapy | (Monotherapy | (Monotherapy |
| escalation) | escalation) | escalation) | escalation) | escalation) |
| N = 2 | N = 4 | N = 7 | N = 6 | N = 19 |

Age (Years), n (%)

| <18 | 0 | | 0 | | 0 | | 0 | | 0 | |
|---|---|---|---|---|---|---|---|---|---|---|
| 18-44 | 0 | | 0 | | 0 | | 0 | | 0 | |
| 45-64 | 1 | (50.0%) | 1 | (25.0%) | 5 | (71.4%) | 1 | (16.7%) | 8 | (42.1%) |
| >=65 | 1 | (50.0%) | 3 | (75.0%) | 2 | (28.6%) | 5 | (83.3%) | 11 | (57.9%) |

| | | | | | |
|---|---|---|---|---|---|
| Median | 65.00 | 68.00 | 62.00 | 72.00 | 70.00 |
| Mean | 65.00 | 67.75 | 63.57 | 72.00 | 67.26 |
| Std Dev | 21.21 | 5.12 | 14.52 | 5.06 | 10.96 |
| Range | (50, 80) | (62, 73) | (48, 90) | (64, 79) | (48, 90) |
| (min, max) | | | | | |

Gender, n (%)

| | | | | | | | | | | |
|---|---|---|---|---|---|---|---|---|---|---|
| Male | 1 | (50.0%) | 3 | (75.0%) | 1 | (14.3%) | 5 | (83.3%) | 10 | (52.6%) |
| Female | 1 | (50.0%) | 1 | (25.0%) | 6 | (85.7%) | 1 | (16.7%) | 9 | (47.4%) |

Race, n (%)

| White | 2 | (100.0%) | 1 | (25.0%) | 3 | (42.9%) | 2 | (33.3%) | 8 | (42.1%) |
|---|---|---|---|---|---|---|---|---|---|---|
| Black or | 0 | | 1 | (25.0%) | 1 | (14.3%) | 0 | | 2 | (10.5%) |
| African | | | | | | | | | | |
| American | | | | | | | | | | |

| Asian | 0 | 0 | | 2 | (28.6%) | 3 | (50.0%) | 5 | (26.3%) |
|---|---|---|---|---|---|---|---|---|---|
| Not reported | 0 | 2 | (50.0%) | 1 | (14.3%) | 1 | (16.7%) | 4 | (21.1%) |

Ethnicity, n (%)

| Hispanic | 0 | | 0 | | 0 | | 1 | (16.7%) | 1 | (5.3%) |
|---|---|---|---|---|---|---|---|---|---|---|
| or Latino | | | | | | | | | | |
| Not | 2 | (100.0%) | 3 | (75.0%) | 4 | (57.1%) | 5 | (83.3%) | 14 | (73.7%) |

| | | | | | | | | |
|---|---|---|---|---|---|---|---|---|
| Hispanic | | | | | | | | |
| or Latino | | | | | | | | |
| Not | 0 | 1 | (25.0%) | 3 | (42.9%) | 0 | 4 | (21.1%) |
| reported | | | | | | | | |

TABLE 5
Demographic Characteristics for Phase I Study

| COMPD A | | |
|---|---|---|
| 5 mg QD + | COMPD A | |
| Fulvestrant | 5 mg QD | |
| (Combination | (Monotherapy | |
| escalation) | expansion) | Total study |
| N = 4 | N = 8 | N = 31 |

Age (Years), n (%)

| <18 | 0 | | 0 | | 0 | |
|---|---|---|---|---|---|---|
| 18-44 | 0 | | 2 | (25.0%) | 2 | (6.5%) |
| 45-64 | 1 | (25.0%) | 5 | (62.5%) | 14 | (45.2%) |
| >=65 | 3 | (75.0%) | 1 | (12.5%) | 15 | (48.4%) |

| | | | |
|---|---|---|---|
| Median | 67.50 | 53.00 | 65.00 |
| Mean | 68.00 | 54.00 | 65.20 |
| Std Dev | 4.24 | 12.51 | 11.67 |
| Range (min, max) | 64, 73) | (39, 76) | (39, 90) |

Gender, n (%)

| Male | 0 | | 0 | | 10 | (32.3%) |
|---|---|---|---|---|---|---|
| Female | 4 | (100.0%) | 8 | (100.0%) | 21 | (67.7%) |

Race, n (%)

| White | 3 | (75.0%) | 3 | (37.5%) | 14 | (45.2%) |
|---|---|---|---|---|---|---|
| Black or African | 0 | | 0 | | 2 | (6.5%) |
| American | | | | | | |
| Asian | 1 | (25.0%) | 5 | (62.5%) | 11 | (35.5%) |

| | | | | |
|---|---|---|---|---|
| Not reported | 0 | 0 | 4 | (12.9%) |

Ethnicity, n (%)

| Hispanic or Latino | 0 | | 0 | | 1 | (3.2%) |
|---|---|---|---|---|---|---|
| Not Hispanic or Latino | 3 | (75.0%) | 6 | (75.0%) | 23 | (74.2%) |
| Not reported | 1 | (25.0%) | 2 | (25.0%) | 7 | (22.6%) |

### Example 2A: Safety and Efficacy Update for Part 1A and Part 1B—Data Cut-Off Date of 30 Sep. 2022

As of the data cut-off date of 30 Sep. 2022, safety and efficacy of COMPOUND A is being assessed in the ongoing, first-in-human, Phase 1 Study described in Example 2. Overall, 29 patients were enrolled; 25 patients in Part 1A (n=12, ER+HER2− breast cancer; n=11, CRPC; n=2, NSCLC) and 4 patients in Part 1B (all ER+HER2− breast cancer). Dose expansion cohorts as monotherapy and in combination with fulvestrant in patients with ER+HER2− breast cancer are ongoing.

### I. Safety Update for Part 1A and Part 1B—Data Cut-Off Date of 30 Sep. 2022

As of 30 Sep. 2022, 25 patients were treated in monotherapy dose escalation, 6 patients at 15 mg QD, 7 patients at 8 mg QD, 4 patients at 5 mg QD, 4 patients at 2 mg QD and 4 patients at 1 mg QD. Four patients were treated in combination dose escalation at 5 mg QD+fulvestrant.

COMPOUND A was well tolerated. The MTD for COMPOUND A was not identified; 5 mg QD was identified as the RDE for both COMPOUND A monotherapy and in combination with fulvestrant.

### Dose Limiting Toxicities (DLTs)

As of 30 Sep. 2022, of the 29 patients dosed with COMPOUND A in Part 1A (monotherapy escalation) and Part 1B (combination escalation), 3 dose limiting toxicities (DLTs) were observed. Of these, 1 DLT was reported at 2 mg QD (monotherapy escalation), 1 DLT was reported at 8 mg QD (monotherapy escalation), and 1 DLT was reported at 5 mg QD+fulvestrant (combination escalation). All 3 DLTs were of Grade 3 neutropenia (decreased neutrophil count): 2 in Part 1A (8 mg and 2 mg QD) and 1 in Part 1B (5 mg QD).

### Adverse Events

Adverse events (AEs) of the patients dosed with COMPOUND A in the Phase 1 clinical trial were coded according to the medical dictionary for regulatory activities (MedDRA), version 24.1. The severity of adverse events were graded according to the National Cancer Institute Common Terminology Criteria for Adverse Events (NCI CTCAE) version 5.0.

### Treatment-Emergent All-Causality Adverse Events (TEAE)

As of 30 Sep. 2022, of the 29 patients dosed with COMPOUND A in Part 1A (monotherapy escalation) and Part 1B (combination escalation), 28 (96.6%) patients experienced at least one treatment emergent adverse event of any grade reported in 20% of patients. The most common TEAEs occurring in 20% of patients were Dysgeusia (N=22, 75.9%); Anemia (N=16, 55.2%); Neutrophil count decreased/Neutropenia (N=14, 48.3%); Diarrhea (N=11, 37.9%); Thrombocytopenia (N=9, 31.0%); Fatigue (N=9, 31.0%); White blood cell count decreased (N=8, 27.6%); Aspartate aminotransferase (AST) increased (N=8, 27.6%); Alanine aminotransferase (ALT) increased (N=6, 20.7%); and Appetite reduction (N=6, 20.7%).

All-causality ≥Grade 3 TEAEs included Neutrophil count decreased/Neutropenia (N=6, 20.7%); Anemia (N=5, 17.2%); White blood cell count decreased (N=2, 6.9%); Diarrhea, Fatigue, Thrombocytopenia, Hypotension, Lymphocyte count decreased and Pneumonitis (N=1, 3.4% each). Of the ≥Grade 3 Neutropenia, 1 (N=6, 16.7%) was reported at 15 mg QD (Part 1A), 2 (N=7, 28.6%) were reported at 8 mg QD (Part 1A), 1 (N=4, 25%) was reported at 5 mg QD (Part 1A), 1 (N=4, 25%) was reported at 2 mg QD (Part 1A) and 1 (N=4, 25%) was reported at 5 mg QD (Part 1B). Of the ≥Grade 3 Anemia, 3 (N=6, 50.0%) were reported at 15 mg QD (Part 1A), 1 (N=7, 14.3%) was reported at 8 mg QD (Part 1A), and 1 (N=4, 25%) was reported at 5 mg QD (Part 1A). Of the ≥Grade 3 White blood cell count decreased, 1 (N=6, 16.7%) was reported at 15 mg QD (Part 1A) and 1 (N=7, 14.3%) was reported at 8 mg QD (Part 1A). Each (N=1, 3.4%) of the ≥Grade 3 Diarrhea, Fatigue, Thrombocytopenia, Hypotension, Lymphocyte count decreased and Pneumonitis were reported at 15 mg QD (Part 1A)

### Treatment-Emergent Treatment Related Adverse Events (TRAE)

As of 30 Sep. 2022, of the 29 patients dosed with COMPOUND A in Part 1A and Part 1B, 27 (93.1%) patients experienced at least one TRAE of any grade reported in 10% of patients. The most common TRAEs occurring in 10% of patients was Dysgeusia (N=21, 72.4%); Anemia (N=15, 51.7%); Neutrophil count decreased/Neutropenia (N=14, 48.2%); Diarrhea (N=9, 31.0%); White blood cell count decreased (N=8, 27.6%); Fatigue (N=7, 24.1); Aspartate aminotransferase (AST) increased (N=6, 20.7%); Thrombocytopenia and Appetite reduction (N=5, 17.2% each); Alanine aminotransferase (ALT) increased, Hypomagnesemia, and Nausea (N=4, 13.8% each); and Vomiting and Lymphocyte count decreased (N=3, 10.3% each).

Across Parts 1A and 1B, TRAEs (any grade) in ≥20% patients were dysgeusia (72%), anemia (52%), neutropenia (48%), thrombocytopenia (31%), diarrhea (31%), white blood cells (WBC) decreased (28%), fatigue (24%), and aspartate aminotransferase increased (21%); the majority of TRAEs were G1-2. TRAEs≥G3 seen in >1 patient were neutropenia ( 6/29; 21%), anemia ( 5/29; 17%), and WBC decreased ( 2/29; 7%).

TRAEs≥Grade 3 include Neutrophil count decreased/Neutropenia (N=6, 20.7%); Anemia (N=5, 17.2%); White blood cell count decreased (N=2, 6.9%); and Diarrhea and Thrombocytopenia (N=1, 3.4% each).

### II. Efficacy

As of 30 Sep. 2022, confirmed and durable clinical responses were observed in heavily treated patients with ER+HER2− breast cancer.

Confirmed and durable partial responses were observed in ⅛ (Part 1A) and 2/4 (Part 1B) response-evaluable pts with ER+/HER2− mBC who progressed on prior ET+CDK4/6 inhibitor treatment.

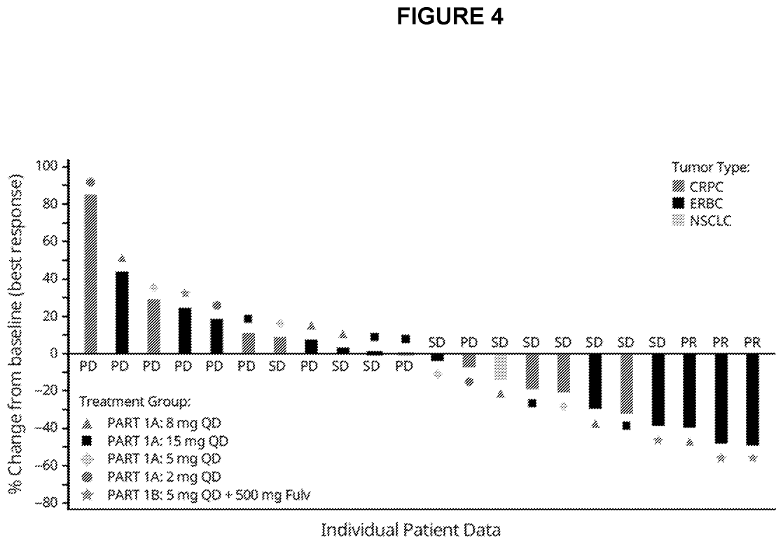

Among response-evaluable patients (n=22), confirmed and durable partial responses (PR) were observed in 3 patients with ER+/HER2− breast cancer, including 1 in Part 1A (8 mg monotherapy) and 2 patients in Part 1B (5 mg QD/fulvestrant 500 mg); (FIG. 4).

Duration of response (DOR): 19.4 months for 1 patient in Part 1A and 8.1 months and 10 months for 2 patients in Part 1B.

Stable disease (SD) was observed in 9 (of 18) patients in Part 1A and 1 (of 4) patient in Part 1B; of these, 5 had ER+/HER2− breast cancer, 4 had CRPC, and 1 had NSCLC.

Three patients had SD lasting ≥6 months; the duration of SD was 11.3 months (patient with CRPC; 15 mg QD), 9.1 months (patient with CRPC; 5 mg QD), and 7.5 months (patient with ER+/HER2− breast cancer; 8 mg QD).

### III. Patient Population

As of 30 Sep. 2022, patient demographic and baseline features across Parts 1A are shown in Table 6.

TABLE 6
Demographic and Baseline Characteristics
(Parts 1A + 1B)(N = 29)

| | | |
|---|---|---|
| Age, median (range), years | 67 | (48-90) |
| Female | 17 | (58.6) |

Race, n (%)

| | | |
|---|---|---|
| White | 14 | (48.3) |
| Black or African American | 3 | (10.3) |
| Asian | 8 | (27.6) |
| Not reported | 4 | (13.8) |

Primary cancer diagnosis, n (%)

| | | |
|---|---|---|
| Breast cancer (ER+/HER2−) | 16 | (55.0) |
| NSCLC | 2 | (7.0 |
| CRPC | 11 | (38.0) |

ECOG PS*, n (%)

| | | |
|---|---|---|
| 0 | 11 | (37.9) |
| 1 | 18 | (62.1) |

Advanced or metastatic breast cancer (ER+/HER2−) prior lines of therapy

| Systemic therapy, median (range) | Part 1A: 7 (1-14) | |
|---|---|---|
| | Part 1B: 5 (3-10) | |
| Prior CDK4/6 inhibitors, n (%) | 15 | (93.8) |
| Prior endocrine therapy, n (%) | 16 | (100.0) |
| Prior chemotherapies, n (%) | 15 | (93.8) |
| Prior targeted therapies (mTOR and PIK), n | 7 | (43.8) |
| (%) | | |

*Eastern Cooperative Oncology Group performance status

Most patients were White (48.3%) or Asian (27.6%); median age was 67 (range: 48-90) years. Among patients with advanced or metastatic ER+/HER2− breast cancer, most (81.3%) received >3 prior lines of systemic anticancer therapy.

A total of 83% (Part 1A) and 75% (Part 1B) of patients with advanced or metastatic ER+/HER2− breast cancer had received >3 prior lines of systemic anticancer therapy. All patients with advanced or metastatic ER+/HER2− breast cancer (n=16) received prior endocrine therapy and 15 patients received prior CDK4/6 inhibitors.

1. A method for treating cancer comprising administering to a subject in need thereof a daily dose of from about 0.1 mg to about 15 mg of a lysine acetyltransferase 6 (KAT6) inhibitor having the structure 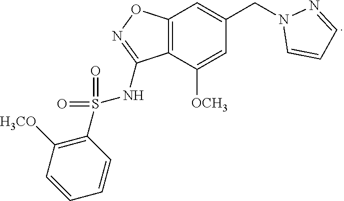 [SMILES: COc1ccccc1S(=O)(=O)Nc1noc2cc(Cn3cccn3)cc(OC)c12]
or a pharmaceutically acceptable salt thereof.

2. A method for treating cancer comprising administering to a subject in need thereof a daily dose of from about 0.1 mg to about 15 mg of 2-methoxy-N-{4-methoxy-6-[(1H-pyrazol-1-yl)methyl]-1,2-benzoxazol-3-yl}benzene-1-sulfonamide, or a pharmaceutically acceptable salt thereof, in combination with
a) an amount of a cyclin-dependent kinase 4 (CDK4 inhibitor);
b) an amount of an antiestrogen; or
c) an amount of a CDK4 inhibitor and an amount of an antiestrogen.

3. The method of claim 1, wherein the daily dose of the KAT6 inhibitor, or the pharmaceutically acceptable salt thereof, is administered once per day (QD).

4. The method of claim 3, wherein the KAT6 inhibitor, or the pharmaceutically acceptable salt thereof, is administered in an amount of from about 0.5 mg to about 5 mg QD.

5. The method of claim 3, wherein the KAT6 inhibitor, or the pharmaceutically acceptable salt thereof, is administered in an amount of from about 0.1 mg to less than 1 mg QD.

6. The method of claim 5, wherein the KAT6 inhibitor, or the pharmaceutically acceptable salt thereof, is administered in an amount of from about 0.1 mg to about 0.75 mg QD.

7. The method of claim 3, wherein the KAT6 inhibitor, or the pharmaceutically acceptable salt thereof, is administered in an amount of about 0.5 mg QD, in an amount of about 1 mg QD, in an amount of about 2 mg QD, in an amount of about 3 mg QD, in an amount of about 4 mg QD, or in an amount of about 5 mg D.

8-12. (canceled)

13. The method of claim 1, wherein the KAT6 inhibitor, or the pharmaceutically acceptable salt thereof, is administered orally.

14. The method of claim 2, wherein the CDK4 inhibitor is a CDK4 selective inhibitor or a CDK4/6 inhibitor.

15. The method of claim 14, wherein the CDK4 inhibitor is a CDK4 selective inhibitor.

16. The method of claim 15, wherein the CDK4 selective inhibitor is 1,5-anhydro-3-({5-chloro-4-[4-fluoro-2-(2-hydroxypropan-2-yl)-1-(propan-2-yl)-1H-benzimidazol-6-yl]pyrmidin-2-yl}amino)-2,3-dideoxy-D-threo-pentitol, or a pharmaceutically acceptable salt thereof.

17. The method of claim 14, wherein the CDK4 inhibitor is a CDK4/6 inhibitor.

18. The method of claim 17, wherein the CDK4/6 inhibitor is abemaciclib, ribociclib or palbociclib, or a pharmaceutically acceptable salt thereof.

19. The method of claim 17, wherein the CDK4/6 inhibitor is or palbociclib, or a pharmaceutically acceptable salt thereof.

20. The method of claim 2, wherein the antiestrogen is an aromatase inhibitor, a selective estrogen receptor degrader (SERD) or a selective estrogen receptor modulator (SERM).

21. The method of claim 20, wherein the antiestrogen is fulvestrant.

22. The method of claim 20, wherein the antiestrogen is letrozole.

23. The method of claim 1, wherein the cancer is breast cancer, lung cancer, or prostate cancer.

24. The method of claim 23, wherein the cancer is breast cancer.

25. The method of claim 24, wherein the breast cancer is ER+HER2− breast cancer.

26. The method of claim 1, wherein the subject is a human.

27. The method of claim 2, wherein the daily dose of the 2-methoxy-N-{4-methoxy-6-[(1H-pyrazol-1-yl)methyl]-1,2-benzoxazol-3-yl}benzene-1-sulfonamide, or the pharmaceutically acceptable salt thereof, is administered once per day (QD).

28. The method of claim 27, wherein the 2-methoxy-N-{4-methoxy-6-[(1H-pyrazol-1-yl)methyl]-1,2-benzoxazol-3-yl}benzene-1-sulfonamide, or the pharmaceutically acceptable salt thereof is administered in an amount of from about 0.5 mg to about 5 mg QD.

29. The method of claim 2, wherein the 2-methoxy-N-{4-methoxy-6-[(1H-pyrazol-1-yl)methyl]-1,2-benzoxazol-3-yl}benzene-1-sulfonamide, or the pharmaceutically acceptable salt thereof is administered in an amount of from about 0.1 mg to less than 1 mg QD.

30. The method of claim 29, wherein the 2-methoxy-N-{4-methoxy-6-[(1H-pyrazol-1-yl)methyl]-1,2-benzoxazol-3-yl}benzene-1-sulfonamide, or the pharmaceutically acceptable salt thereof is administered in an amount of from about 0.1 mg to about 0.75 mg QD.

31. The method of claim 27, wherein the 2-methoxy-N-{4-methoxy-6-[(1H-pyrazol-1-yl)methyl]-1,2-benzoxazol-3-yl}benzene-1-sulfonamide, or the pharmaceutically acceptable salt thereof is administered in an amount of about 0.5 mg QD, in an amount of about 1 mg QD, in an amount of about 2 mg QD, in an amount of about 3 mg QD, in an amount of about 4 mg QD, or in an amount of about 5 mg QD.

32. The method of claim 2, wherein the 2-methoxy-N-{4-methoxy-6-[(1H-pyrazol-1-yl)methyl]-1,2-benzoxazol-3-yl}benzene-1-sulfonamide, or the pharmaceutically acceptable salt thereof is administered orally.

33. The method of claim 2, wherein the cancer is breast cancer, lung cancer, or prostate cancer.

34. The method of claim 33, wherein the cancer is breast cancer.

35. The method of claim 34, wherein the breast cancer is ER+HER2− breast cancer.

36. The method of claim 2, wherein the subject is a human.
